(19) United States
(12) Patent Application Publication
(10) Pub. No.: US 2010/0331544 A1
(43) Pub. Date: Dec. 30, 2010

(54) METHOD FOR PRODUCING (1R,5S) ANHYDROECGONINE ESTER SALTS

(75) Inventors: Carsten H. Puder, Ingelheim am Rhein (DE); Thomas Hoellmueller, Gau-Algesheim (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM PHARMA GMBH & CO. KG, Ingelheim am Rhein (DE)

(21) Appl. No.: 12/680,066

(22) PCT Filed: Sep. 25, 2008
(86) PCT No.: PCT/EP08/62839

§ 371 Date: Jul. 12, 2010

(30) Foreign Application Priority Data
Sep. 28, 2007 (DE) 102007046887.5

(51) Int. Cl. C07D 451/02

(52) U.S. Cl. 546/132

(57) ABSTRACT

The invention relates to a large-scale method for producing salts of (IR,5S) anhydroecgonine esters. The salt formation and selective crystallization of (IR,5S) anhydroecgonine esters with chiral acids is highly efficient in producing an enantiomer form, any undesired enantiomers and other impurities being removed. The ester and its salts are used as the starting material for producing active agents.

### TECHNICAL FIELD OF THE INVENTION

The invention relates to a process for preparing salts of (1R,5S)-anhydroecgonin ester. The process according to the invention is particularly suitable for the large-scale manufacture of these salts with a high degree of enantiomeric purity with respect to the (1R,5S)-anhydroecgonin esters.

### BACKGROUND TO THE INVENTION

The (1R,5S)-anhydroecgonin esters on which the present invention is based are easily characterisable active substance precursors having the following general chemical formula 5:

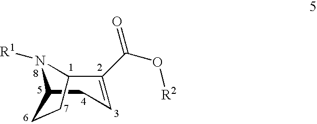

wherein

R¹ denotes hydrogen, an alkyl group, preferably methyl, ethyl, propyl or butyl, or any desired protective group, preferably allyl, benzyl, methoxybenzyl, allyloxycarbonyl, benzyloxycarbonyl, tert.-butyloxycarbonyl, 9-fluorenylmethyloxycarbonyl, acetyl, benzoyl or formyl;

R² denotes alkyl, aryl, preferably phenyl or naphthyl, optionally substituted by one or more substituents selected from halogen, hydroxy, amino, cyano, nitro, trifluoromethyl, trifluoromethoxy, alkoxy, cycloalkoxy, alkyl, cycloalkyl, cycloalkylalkyl, alkenyl and alkynyl; R² is preferably methyl, ethyl, propyl or butyl.

(1R,5S)-Anhydroecgonin esters are used as starting materials for the preparation of pharmaceutical active substances. The 8-azabicyclo[3.2.1]oct-2-ene system, which derives from tropane as the basic structure, constitutes a monounsaturated heterocyclic ring system in which the 1st and 5th C atom of a piperidine ring are joined together by an ethylene group. For example systems of this kind play a part as starting or intermediate products for pharmaceutically active tropane derivatives. These systems are of importance, for example, in connection with potent and selective ligands of the nicotinic acetylcholine receptors (nAChR). The hope is that such ligands will have a favourable influence on certain diseases, for example dementia, such as senile dementia, Alzheimer's disease, Parkinson's disease or cognitive disorders, such as depression and psychoses.

Numerous methods by which these (1R,5S)-anhydroecgonin esters can be prepared are known from the prior art:

One possibility is to use cocaine, such as for example cocaine hydrochloride, as the starting material and to react it with an alkoxide to form the enantiomerically pure (1R,5S)-anhydroecgonin ester. This so-called cocaine route is described for example in Patent Application WO 96/30371 A1 and is illustrated in the following reaction equation for preparing anhydroecgonin ethyl ester:

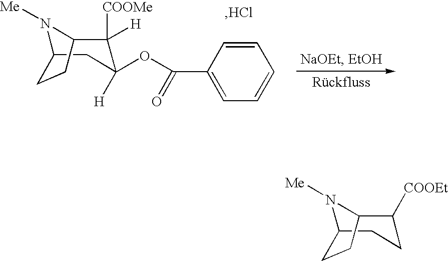

This circumvents the need for racemate cleavage by using the desired absolute configuration as the starting material.

The disadvantages of using cocaine as the starting material for a synthesis are obvious: the world market for controlled, i.e. legally obtained, cocaine, is small and the number of suppliers is limited. As a result the prices for cocaine hydrochloride, for example, are very high and the synthesis of (1R,5S)-anhydroecgonin esters via the cocaine route is correspondingly expensive. Moreover, cocaine is covered by the drugs laws that regulate general handling of narcotics, with the result that special dispensation is needed from the Federal Institute for Pharmaceuticals and Medical Products (BfArM) in order to acquire it.

Therefore, it is preferable to carry out synthesis, particularly total synthesis, of the anhydroecgonin system which does not require cocaine as starting material.

For example, S. P. Findlay, J. Org. Chem. 1957, 22, 1385-1394, describes total synthesis of the picrate of racemic anhydroecgonin methyl ester, in which the precursor 2-carbomethoxytropinone is prepared starting from ketoglutaric anhydride, methylamine and succindialdehyde. Racemate cleaving of the 2-carbomethoxytropinone using (2R,3R)-tartaric acid (L-tartaric acid) via the hydrogen tartrate is described.

In addition, WO 2004/072071 A1 describes the reduction of a carbomethoxytropinone base by means of sodium borohydride and reaction with sodium ethoxide in ethyl ester to form the anhydroecgonin ethyl ester. In WO 2004/072071 A1, there is no mention of the enantiomeric purity of the base used and of the anhydroecgonin ethyl ester obtained therefrom.

A disadvantage of the methods of synthesis described in the prior art is that they are not designed for use on an industrial scale and do not satisfy the particular requirements of mass production.

There is very little information in the prior art on the formation of salts of (1R,5S)-anhydroecgonin ester. Reference may be made to R. C. Bick et al., Aust. J. Chem. 1979, 32, 2537-2543 and C. Grundmann et al., Liebigs Ann. Chem. 1957, 605, 24-32, by way of example. Both publications briefly describe the preparation of the picrate in the experimental section. As picric acid is not chiral, it is not possible to achieve a depletion in this manner by the formation of diastereomeric salts. Theoretically, depletion is only possible by controlling the yield.

The publication by C. Grundmann et al. additionally describes the preparation of (1R,5S)-anhydroecgonin ethyl ester-(2′S,3′S)-dibenzoylhydrogen tartrate. The manufacturing instructions published by C. Grundmann et al. are very complicated and the solvent consumption is extremely high, i.e. the racemate cleaving as described could not reasonably be converted into a commercial operation.

The publication by C. Grundmann et al. further describes how, except by using picric acid as already mentioned, the authors have not succeeded in separating the antipodes using L-malic acid, D-tartaric acid [(2S,3S)-tartaric acid], d-10-camphorsulphonic acid [(1S,4R)-camphor-10-sulphonic acid] and 3-bromo-d-camphorsulphonic acid-7. Thus, on page 27 it says: “The cleaving of the synthetic anhydroecgonin ethyl ester into the two optical antipodes comes up against serious difficulties. L-malic acid, D-tartaric acid, d-10-camphorsulphonic acid, 3-bromo-d-camphorsulphonic acid-7 are not suitable, as they all produce only salts that crystallise poorly.”

Surprisingly, this has been refuted by the present invention.

Moreover, although WO 96/30371 A1 does mention salts of anhydroecgonin esters, this is only in connection with the separation of racemates. According to the invention, however, the salts are used to separate a mixture of enantiomers in which the desired enantiomer is always significantly preponderant.

Thus, in the prior art there has not hitherto been any efficient method suitable for scaling up for the preparation of (1R,5S)-anhydroecgonin esters and the salts thereof.

The aim of the present invention is thus to provide an improved method of synthesis, particularly for use on an industrial scale, providing a method of synthesising the anhydroecgonin esters or the salts thereof. A further aim is to provide an economical process that is sparing of resources and capable of being scaled up, with the aim of depleting the unwanted enantiomers and other impurities to an optimum level.

### DESCRIPTION OF THE INVENTION

According to the invention a process is provided that is suitable for large-scale industrial production of enantiomerically pure salts of (1R,5S)-anhydroecgonin ester according to the teachings of the claims. The salt formation and selective crystallisation of (1R,5S)-anhydroecgonin esters with chiral acids leads with great efficiency to a largely enantiomerically pure form, while any unwanted enantiomers and other impurities present are depleted. The ester and the salts thereof are used as starting material for the preparation of active substances.

In a first aspect the present invention starts from (1R,5S)-anhydroecgonin esters of general chemical formula 5:

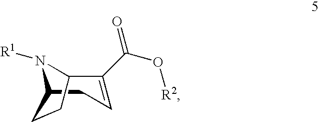

wherein

R¹ denotes hydrogen, an alkyl group, preferably methyl, ethyl, propyl or butyl, or any desired protective group, preferably allyl, benzyl, methoxybenzyl, allyloxycarbonyl, benzyloxycarbonyl, tert.-butyloxycarbonyl, 9-fluorenylmethyloxycarbonyl, acetyl, benzoyl or formyl;

R² denotes alkyl, aryl, preferably phenyl or naphthyl, optionally substituted by one or more substituents selected from halogen, hydroxy, amino, cyano, nitro, trifluoromethyl, trifluoromethoxy, alkoxy, cycloalkoxy, alkyl, cycloalkyl, cycloalkylalkyl, alkenyl and alkynyl; preferably R² is methyl, ethyl, propyl or butyl.

Suitable protective groups for R¹ may be found in the prior art, e.g. Theodora W. Green, Peter G. M. Wuts, Protective Groups in Organic Chemistry, John Wiley, 3rd edition.

The process for preparing the neutral anhydroecgonin esters largely adheres to the remarks made in the prior art or is analogous thereto. Regarding steps (1a) to (3) listed below reference is made to S. P. Findlay, J. Org. Chem. 1957, 22, 1385-1394, particularly variant F, examples analogous to step (4) can be found in the literature and with regard to the subsequent step (5) reference is made to WO 2004/072071, page 16, Method A.

Compounds on which the present invention is based in which the substituents R¹ and R² deviate from those prescribed in the above-mentioned prior art are prepared analogously.

The general manufacturing method for preparing the (1R,5S)-anhydroecgonin esters is summarised below [process steps (1a) to (5)]. The salt formation according to the invention is then described. Process steps (3) and (4) are optional.

(1a) Reaction of a compound of formula 1 with methanol to form a compound of formula 1′:

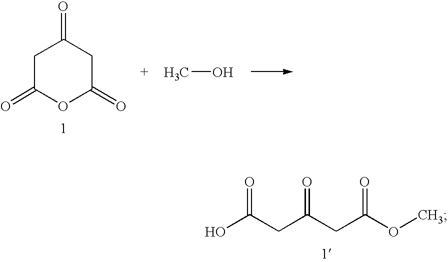

(1b) Reaction of a compound of formula 2 with water and catalyst (sulphuric acid) to form a compound of formula 2′:

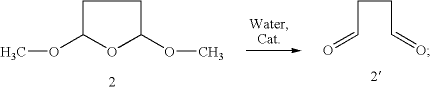

(2) Reaction of a compound of formula 1′ and a compound of formula 2′ with R¹-amine to form a compound of formula 3:

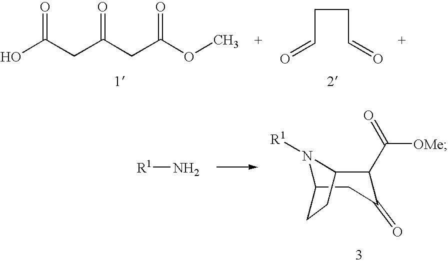

wherein R¹ denotes hydrogen, an alkyl group, preferably methyl, ethyl, propyl or butyl, or any desired protective group, preferably allyl, benzyl, methoxybenzyl, allyloxycarbonyl, benzyloxycarbonyl, tert.-butyloxycarbonyl, 9-fluorenylmethyloxycarbonyl, acetyl, benzoyl or formyl, (R¹-amine is preferably an amine selected from among methylamine, ethylamine, propylamine and butylamine);

(3) Reaction of the compound of formula 3 with (2R,3R)-tartaric acid [L(+)-tartaric acid] with enrichment of the (1R,5S)-enantiomer-hydrogen tartrate salt of the compound of formula 3′:

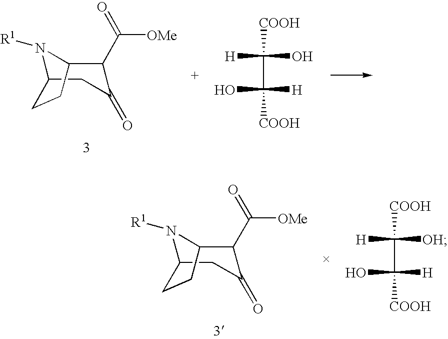

(4) The largely enantiomerically pure base 4 may optionally be prepared from compound 3′ in conventional manner.

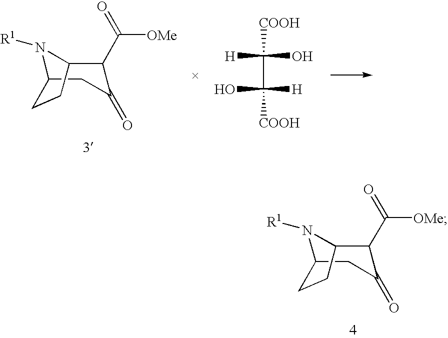

(5) Reduction of the compound of formula 4 analogously to WO 2004/072071 and optionally subsequent transesterification with the corresponding alkoxide MOR² using methods known from the literature.

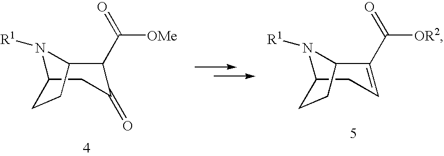

wherein R² denotes alkyl, aryl, preferably phenyl or naphthyl, optionally substituted by one or more substituents selected from halogen, hydroxy, amino, cyano, nitro, trifluoromethyl, trifluoromethoxy, alkoxy, cycloalkoxy, alkyl, cycloalkyl, cycloalkylalkyl, alkenyl and alkynyl, R² preferably denotes methyl, ethyl, propyl or butyl, M denotes an alkali or alkaline earth metal, preferably potassium or sodium;

(6) Reaction of the compound of formula 5 with a chiral acid to form a salt of the compound of formula 5′;

(7) optionally further purification of the chiral compound of formula 5′ using separation methods known from the literature and

(8) optionally crystallisation.

The reaction according to step (6) according to the invention takes place according to one of the following chemical equations:

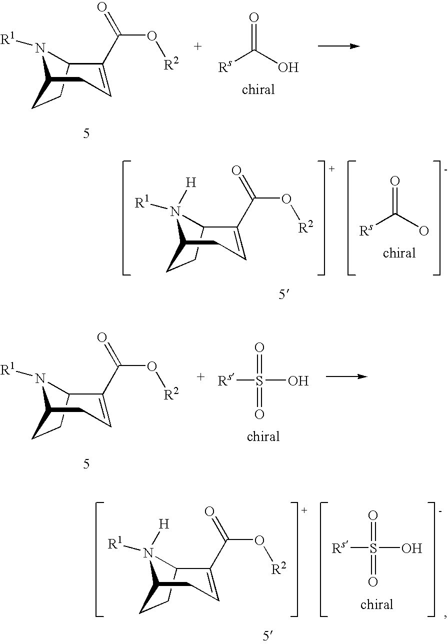

wherein R¹ and R² are as hereinbefore defined and R^{s} and R^{s′} denote the acid groups of the chiral acids used.

In a second aspect the invention also relates to salts of the compound of formula 5′:

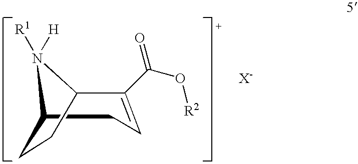

with X⁻:

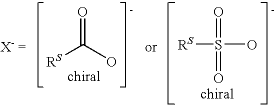

In another aspect the present invention relates to a process according to steps (1), (2), (5) and (6), optionally including steps (3) and (4).

### DETAILED DESCRIPTION OF THE INVENTION

Definitions of Terms

The term “aryl” or “aryl group” denotes a 6- to 10-membered aromatic carbocyclic group and includes for example phenyl and naphthyl. Other terms that contain the term aryl as a component have the same meaning for the aryl component. Examples of these components are: arylalkyl, aryloxy or arylthio.

By the terms “alkyl” or “alkyl groups” as well as alkyl groups which are a part of other groups are meant branched and unbranched alkyl groups with 1 to 6 carbon atoms. The following are mentioned by way of example: methyl, ethyl, propyl, butyl, pentyl, hexyl. Unless otherwise stated, the above-mentioned terms propyl, butyl, pentyl and hexyl include all the possible isomeric forms. For example the term propyl includes the two isomeric groups n-propyl and iso-propyl, the term butyl includes the isomers groups n-butyl, iso-butyl, sec. butyl and tert.-butyl.

By “alkoxy” or “alkyloxy groups” are meant branched and unbranched alkyl groups with 1 to 6 carbon atoms which are linked by an oxygen atom. The following are mentioned by way of example: methoxy, ethoxy, propoxy, butoxy, pentoxy, hexoxy. Unless otherwise stated, the above-mentioned terms include all the possible isomeric forms.

Alkenyl groups represent branched and unbranched alkenyl groups with 2 to 6 carbon atoms, preferably 2 to 4 carbon atoms, which have at least one double bond, such as for example the above-mentioned alkyl groups, provided that they have at least one double bond in the molecule, for example vinyl, propenyl, isopropenyl, butenyl, pentenyl and hexenyl.

Alkenylene groups are branched and unbranched alkenyl bridges with 2 to 6 carbon atoms, preferably 2 to 4 carbon atoms with at least one double bond in the molecule, e.g. the above-mentioned alkylene groups, provided that they have at least one double bond, such as for example vinylene, propenylene, isopropenylene, butenylene, pentenylene and hexenylene.

Unless otherwise specified, the above-mentioned alkenyl and alkenylene groups should be understood as including any stereoisomers that exist. Accordingly, for example, the definition 2-butenyl should be understood as including 2-(Z)-butenyl and 2-(E)-butenyl etc.

The term alkynyl groups relates to alkynyl groups with 2 to 6, preferably 2 to 4 carbon atoms, provided that they have at least one triple bond in the molecule, e.g. ethynyl, propargyl, butynyl, pentynyl and hexynyl.

Halogen denotes fluorine, chlorine, bromine or iodine, preferably chlorine or bromine.

The terms “carbocyclic ring” or “cycloalkyl groups” denote cycloalkyl groups having 3 to 6 carbon atoms, for example cyclopropyl, cyclobutyl, cyclopentyl or cyclohexyl.

The term “nitrogen” as well as the corresponding element symbol includes every oxidised form thereof and quaternary forms of a basic nitrogen atom should also be included.

In specific embodiments of the invention the term “approximately” indicates within 20%, preferably within 10% and more preferably within 5% of a given value or range. A given range of values includes and discloses all the values and intervals contained within it.

If a chemical formula should contradict a chemical name and the skilled man is not immediately able to clear up the contradiction using his specialist knowledge and capabilities, in case of doubt the formula should be taken as authoritative.

### Preferred Embodiments

The process according to the invention will now be described in detail. The manufacture of the starting product 5 needed for the salt formation according to the invention is illustrated in the following reaction plan 1:

### Reaction Plan 1: Total Synthesis of Anhydroecgonin Esters:

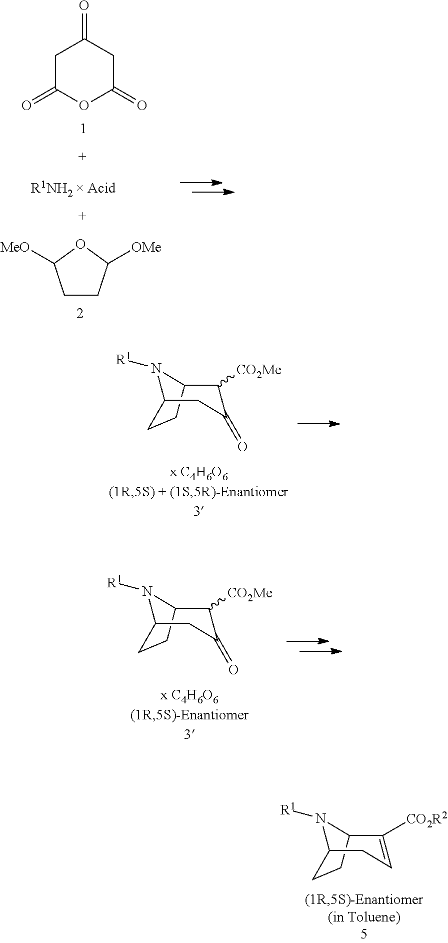

Me denotes methyl.

As already mentioned the synthesis of 5 takes place according to the prior art, particularly S. P. Findlay, J. Org. Chem. 1957, 22, 1385-1394, particularly process variant F, and WO 2004/072071, page 16, method A.

The reaction of the compound of formula 5 according to the invention to form the salt may take place immediately after step (5). For this, in step (6) a compound of formula 5 is reacted with a chiral acid to form a salt of the compound of formula 5′ according to the following chemical equations:

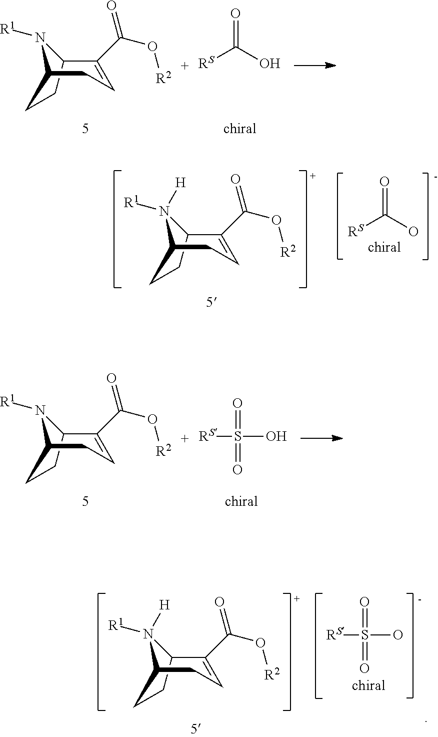

The crystallisation of compounds of formula 5 (1R,5S)-anhydroecgonin ester with optically active acids is not a straightforward matter. Numerous chiral acids have been investigated under different conditions for their ability to crystallise with the compound of formula 5. For the salt formation the compound of formula 5 is initially in dissolved form in a solvent, preferably in concentrated form (“concentrate”). By the expressions “in concentrated form” or “concentrate” is meant a solution of at least 20 wt. %, preferably at least 30 wt. %, more preferably at least 40 wt. %, more preferably at least 50 wt. % and even more preferably at least 60 wt. %. The solvents used are organic solvents with a boiling point below 150° C. (at p=1 bar), preferably toluene, xylene (all the isomers), halobenzenes, aliphatic hydrocarbons (C₅ to C₈), halogen-containing aliphatic hydrocarbons (C₁ to C₆), aliphatic ethers (C₄ to C₈), esters of formic acid (C₂ to C₇), esters of acetic acid (C₃ to C₇) or nitriles (C₂ to C₅).

Most preferred are aromatic hydrocarbons. Examples include toluene and xylene. Most preferred is toluene. If the compound of formula 5 is not supposed to dissolve initially in the crystallisation solvent, it may be dissolved beforehand in a different suitable solvent, for example an alcohol such as methanol or ethanol, so as to make the desired concentrate obtainable.

The following is a summary by way of example of some of the particularly preferred conditions for carrying out salt formation:

The salt formation is preferably carried out in a toluene solution. The compound 5 is dissolved in toluene, while if desired the compound may have already been dissolved in a different solvent, for example the solvent from the preceding reaction step. Preferably the compound 5 is initially dissolved in an alcohol, for example methanol or ethanol. Preferably the chiral acid is placed in a solvent and to this is added the toluene concentrate of the compound of formula 5, preferably with stirring. The salt formation makes it possible to separate any enantiomeric mixtures that may be present, so that the product, the (1R,5S)-enantiomer, can be separated off in an enantiomeric purity of above about 95%, more preferably above about 98%, particularly above about 99%, most preferably above about 99.9%.

Examples of chiral acids that may be used are: (1S,4R)-camphor-10-sulphonic acid, (1R,4S)-camphor-10-sulphonic acid, (2R,3R)-di-p-toluoyltartaric acid, (2S,3S)-di-p-toluoyltartaric acid, (2R,3R)-tartaric acid, (2S,3S)-tartaric acid, (2R,3R)-dibenzoyltartaric acid and (2S,3S)-dibenzoyltartaric acid.

Surprisingly, contrary to the views expressed in the prior art (cf. Supra, C. Grundmann et al.) it was possible to obtain a salt with (1S,4R)-camphor-10-sulphonic acid (=d-10-camphorsulphonic acid). The camphor-10-sulphonate crystallised in a high quality, while the unwanted (1S,5R)-enantiomer was very easily depleted. The yield can be optimised accordingly, for example by controlled slowing down of the crystallisation process, working at lower temperatures at the end of the crystallisation process or the like.

Specifically, in order to prepare the salts of the compound of formula 5′, first of all the chiral acid is preferably dissolved in a solvent, preferably acetone, at elevated temperature, for example 35 to 60° C., preferably about 40 to 50° C., in individual cases while heating to the reflux temperature of the solvent. Instead of acetone, it is also possible to use, as the solvent, alcohols (C₁ to C₅), nitriles (C₂ to C₃) and ketones (C₃ to C₆). All polar and medium-polar protic or aprotic solvents with and without the addition of varying amounts of H₂O are generally possible. This depends on the acid used in each case. In individual cases it may be useful to filter the solution obtained while it is still hot.

Then the compound of formula 5, optionally dissolved in one of the above mentioned solvents, is added to the solution of the chiral acid, preferably with stirring. The educt used may be the compound of formula 5 with a variable content of solvent, while advantageously the toluene concentrate obtained in step (5) is used directly, optionally with the addition of another solvent. The compound of formula 5 may be used as a mixture of enantiomers which also contains, in addition to the desired enantiomer [(1R,5S)-enantiomer], an amount of the unwanted enantiomer [(1S,5R)-enantiomer]. For example it is possible to use the compound of formula 5 with an enantiomeric purity ≧80%, preferably ≧90% of the desired enantiomer [(1R,5S)-anhydroecgonin ester] and corresponding amounts of the unwanted enantiomer.

It is particularly preferable for the solution of the chiral acid still to be at elevated temperature, more preferably at or around the temperature at which the chiral acid was dissolved, while the compound of formula 5 is added. This means that, as far as possible, the temperature during the addition of the compound of formula 5 is only about 20° C., preferably about 10° C., still more preferably about 5° C. below the solution temperature for the chiral acid.

The solution obtained may optionally be cooled after heating to the reflux temperature of the solvent, after which, optionally after inoculation with a small amount of seed crystals and/or trituration, the desired enantiomer is precipitated as a salt with an enantiomeric purity ≧98%, preferably ≧99%, more preferably ≧99.9%. The final temperature of the (1R,5S)-enantiomer during the precipitation is, particularly preferably, between −15 and 35° C., particularly 5 to 35° C. The precipitation of the salt from the solvent is particularly preferably carried out at dilutions (Σ m_{educts}: Σ Vₛₒₗᵥₑₙₜ) of 1:10 to 2:1.

After suitable separation, an enantiomeric purity of ≧99.9% of the (1R,5S)-enantiomer in the form of the salt may be achieved by corresponding working up, such as washing with solvent and further purification, for example by chromatographic processes and the like.

In contrast to the procedures known from the prior art (C. Grundmann et al.), which cannot be transferred to the industrial scale, this is possible with the process according to the invention.

The toluene concentrate may be used directly, without a step of preparation or working up, such as elimination of the toluene in vacuo, for example, or a starting material from a commercial source in the form of a mixture of enantiomers may also be used, while by depletion of one of the two enantiomers the desired enantiomer is obtained as a salt with an enantiomeric purity of ≧98%, preferably ≧99%, particularly preferably ≧99.9%.

The invention also relates to the salts of the compound of formula 5′ with a chiral acid. The chiral acid is preferably selected from: (1S,4R)-camphor-10-sulphonic acid, (1R,4S)-camphor-10-sulphonic acid, (2R,3R)-di-p-toluoyltartaric acid, (2S,3S)-di-p-toluoyltartaric acid, (2R,3R)-tartaric acid, (2S,3S)-tartaric acid, (2R,3R)-dibenzoyltartaric acid and (2S,3S)-dibenzoyltartaric acid. The following are examples of chiral acids:

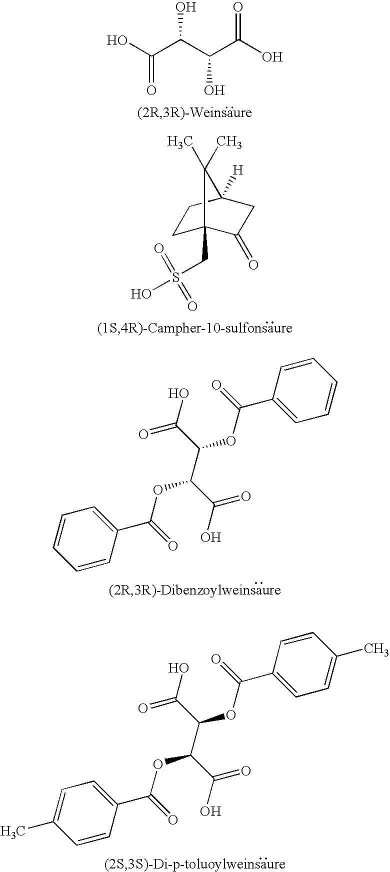

The chiral acids are obviously not limited to the chiral acids mentioned. The skilled man will know of other chiral acids that can also be used to prepare salts of the compound of formula 5′.

According to the invention, salts of the following esters are most particularly preferred: (1R,5S)-anhydroecgonin ethyl ester, (1R,5S)-anhydroecgonin methyl ester, (1R,5S)-anhydroecgonin propyl ester and (1R,5S)-anhydroecgonin butyl ester.

The following is a (1R,5S)-anhydroecgonin ethyl ester that is particularly preferred according to the invention:

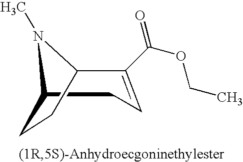

Particularly preferably, the salt of the compound of formula 5′ is one of the following compounds:

(1R,5S)-anhydroecgonin ethyl ester-(1′S,4′R)-camphor-10-sulphonate;

(1R,5S)-anhydroecgonin ethyl ester-(1′R,4′S)-camphor-10-sulphonate;

(1R,5S)-anhydroecgonin ethyl ester-(2′S,3′S)-di-p-toluoylhydrogen tartrate;

(1R,5S)-anhydroecgonin ethyl ester-(2′R,3′R)-di-p-toluoylhydrogen tartrate;

(1R,5S)-anhydroecgonin ethyl ester-(2′R,3′R)-hydrogen tartrate-monohydrate;

(1R,5S)-anhydroecgonin ethyl ester-(2′S,3′S)-hydrogen tartrate-monohydrate;

(1R,5S)-anhydroecgonin ethyl ester-(2′R,3′R)-dibenzoylhydrogen tartrate;

(1R,5S)-anhydroecgonin ethyl ester-(2′S,3′S)-dibenzoylhydrogen tartrate.

Surprisingly a readily crystallisable monohydrate was prepared from (2R,3R)-tartaric acid with (1R,5S)-anhydroecgonin ethyl ester in the presence of water. This is the first isolated monohydrate of an anhydroecgonin ethyl ester salt.

The use of salts of the compound of formula 5′ has a number of advantages over the use of the toluene concentrate of compound 5:

Thus, with the toluene concentrate, the content of nitrogen bases cannot be constantly adjusted without major analytical or operational input. According to experiments, this value fluctuates in prepared charges between 65 and 95 wt. %, so that the further processing of the toluene concentrate may give rise to problems.

The toluene concentrate cannot be purified further, in contrast to the salt. In theory the toluene concentrate can be distilled in vacuo, but laboratory tests have shown that in spite of generous cutting of the main fraction there is only a slight improvement with respect to the chromatographic purity. It was only possible to separate off the solvent, but no significant purification could be achieved. By contrast, the salt may be purified by recrystallisation, for example.

Moreover the stability of the compound of formula 5 is generally less in solution than in solid form, i.e. as a crystallised salt. This affects the shelf life, durability and storage, which is not a long-term possibility for the dissolved form.

In addition, the transportation and storage of solids is generally safer and easier than the transportation of liquids, which take up more space and have to be transported in suitably fluidtight containers.

From the point of view of appearance as well, the solid has advantages over the solution of the products. Thus, the toluene concentrate is obtained as an brownish-orange solution whereas the salts are generally isolated in the form of a white solid.

Furthermore, salts of the anhydroecgonin esters per se may be characterised more precisely and more simply than solutions or concentrates of the anhydroecgonin esters.

Compared with the prior art, particularly the procedure laid down by C. Grundmann et al., a significantly higher yield is obtained. The process according to the invention is more sparing of resources, as the quantity of solvent can be reduced to a fraction and the process is thus cheaper.

The totally synthetic production method according to the invention avoids the use of cocaine hydrochloride as educt; however, the product is still obtained efficiently with a very high ee value, thus holding out the prospect of providing large amounts of pure (1R,5S)-anhydroecgonin ester and/or the salts thereof as starting materials for the preparation of active substances.

To summarise, the invention may be described as follows:

A first aspect 1 of the present invention relates to a process for preparing chiral salts of (1R,5S)-anhydroecgonin esters of general chemical formula 5, which are preferably obtained in an enantiomer-enriched form:

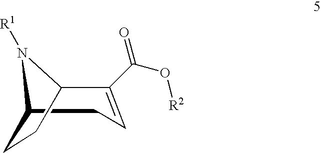

wherein

R¹ denotes hydrogen, an alkyl group, preferably methyl, ethyl, propyl or butyl, or any desired protective group, preferably allyl, benzyl, methoxybenzyl, allyloxycarbonyl, benzyloxycarbonyl, tert.-butyloxycarbonyl, 9-fluorenylmethyloxycarbonyl, acetyl, benzoyl or formyl;

R² denotes alkyl, aryl, preferably phenyl or naphthyl, optionally substituted by one or more substituents selected from halogen, hydroxy, amino, cyano, nitro, trifluoromethyl, trifluoromethoxy, alkoxy, cycloalkoxy, alkyl, cycloalkyl, cycloalkylalkyl, alkenyl and alkynyl; preferably R² is methyl, ethyl, propyl or butyl

with the steps of

(1a) Reaction of a compound of formula 1 with methanol and optionally in methanol as solvent to form a compound of formula 1′:

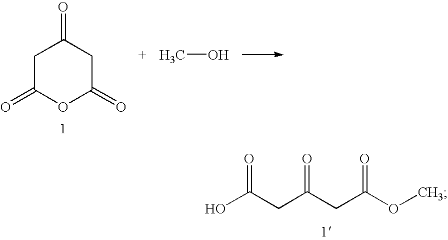

(1b) Reaction of a compound of formula 2 with water and catalyst to form a compound of formula 2′

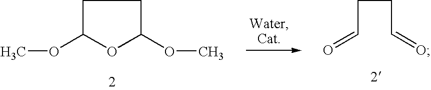

(2) Reaction of a compound of formula 1′ and a compound of formula 2′ with an R¹-amine solution to form a compound of formula 3:

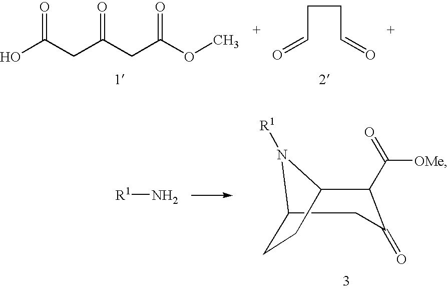

wherein R¹ denotes hydrogen, an alkyl group, preferably methyl, ethyl, propyl or butyl, or any desired protective group, preferably allyl, benzyl, methoxybenzyl, allyloxycarbonyl, benzyloxycarbonyl, tert.-butyloxycarbonyl, 9-fluorenylmethyloxycarbonyl, acetyl, benzoyl or formyl;

(3) optionally reacting the compound of formula 3 with (2R,3R)-tartaric acid [L(+)-tartaric acid] while enriching the (R)-enantiomer-hydrogen tartrate salt of the compound of formula 3′:

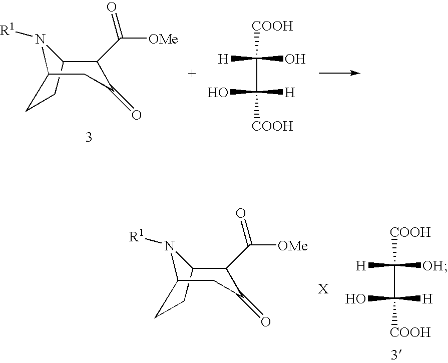

(4) optionally releasing the compound 4 in the basic

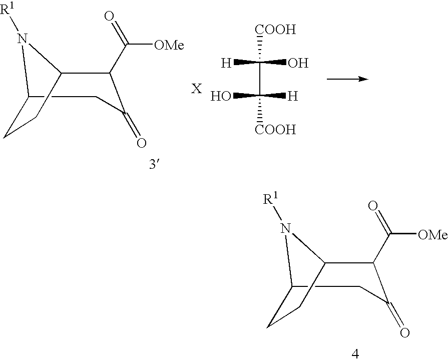

(5) reduction of the product obtained and optionally further transesterification with an alkoxide of formula MOR² to form a compound of formula 5:

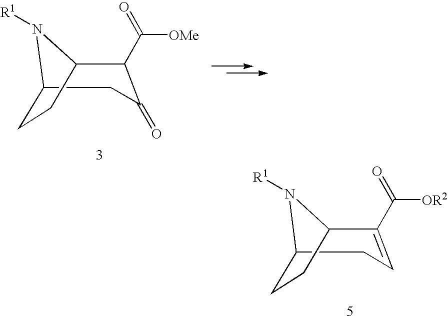

wherein R² denotes alkyl, aryl, preferably phenyl or naphthyl, optionally substituted by one or more substituents selected from halogen, hydroxy, amino, cyano, nitro, trifluoromethyl, trifluoromethoxy, alkoxy, cycloalkoxy, alkyl, cycloalkyl, cycloalkylalkyl, alkenyl and alkynyl, R² preferably denotes methyl, ethyl, propyl or butyl, M denotes an alkali or alkaline earth metal, preferably potassium or sodium; and

(6) reacting the compound of formula 5 with a chiral acid to form a salt of the compound of formula 5′:

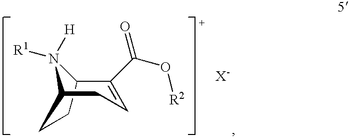

wherein X⁻ denotes the anion of a chiral acid;

(7) optionally purifying the chiral compound of formula 5′ and

(8) optional crystallisation.

In the process according to Aspect 1 of the invention, at the end of the working up in step (5) a concentrated toluene solution of the compound of formula 5 may be prepared by the addition of toluene (Variant 2).

In the process according to Aspect 1 of the invention in step (6) the compound of formula 5 may be present as a concentrate dissolved in toluene in an amount of at least 20 wt. %, preferably at least 40 wt. %, particularly preferably at least 60 wt. % (Variant 3).

In the process according to Aspect 1 of the invention in step (6) the chiral acid may be placed in a solvent and to this is added the concentrated toluene solution of the compound of formula 5 (Variant 4).

In the process according to Aspect 1 of the invention and Variants 2, 3 and 4 thereof, the chiral acid may be selected from among: (1S,4R)-camphor-10-sulphonic acid, (1R,4S)-camphor-10-sulphonic acid, (2R,3R)-di-p-toluoyltartaric acid, (2S,3 S)-di-p-toluoyltartaric acid, (2R,3R)-tartaric acid, (2S,3S)-tartaric acid, (2R,3R)-dibenzoyltartaric acid and (2S,3S)-dibenzoyltartaric acid (Variant 5).

In the process according to Aspect 1 of the invention the solvent in step (6) may be selected from among polar or medium-polar protic or aprotic solvents, preferably acetone, C₁- to C₅-alcohols, C₂- to C₃-nitriles, C₃- to C₆-ketones, with or without the addition of water (Variant 6).

In the process according to Aspect 1 of the invention the chiral acid may be dissolved in the solvent in step (6) with heating to a temperature in the range from about 35° C. to approximately the reflux temperature of the solvent used (Variant 7).

In the process according to Aspect 1 of the invention the concentrated toluene solution of the compound of formula 5 may be added to the solution of the chiral acid in step (6) at or close to the dissolution temperature of the chiral acid (Variant 8).

In the process according to Aspect 1 of the invention and also in Variants 7 or 8 thereof cooling may be carried out in step (6) after the addition of the concentrated toluene solution and optional heating to the reflux temperature of the solvent (Variant 9).

In the process according to Aspect 1 of the invention the compound of formula 5′ may be precipitated at a final temperature of between −15 and 35° C., preferably 5 to 35° C. (Variant 10).

In the process according to Aspect 1 of the invention the precipitation may be assisted by inoculation with a small amount of seed crystals and/or trituration (Variant 11).

In the process according to Aspect 1 of the invention, in step (6), the compound of formula 5′ may be precipitated in the form of an enantiomer in an enantiomeric purity of more than about 95%, preferably more than about 96%, particularly preferably more than about 98%, particularly more than about 99%, most particularly preferably more than about 99.9% (Variant 12).

In a second aspect the invention relates to a process for preparing salts of the compound of formula 5:

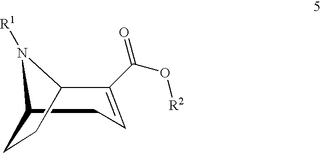

in which the compound of formula 5 is reacted with a chiral acid to obtain a salt of the compound of formula 5′:

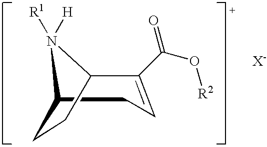

wherein R¹ and R² are defined as in claim 1, and

X⁻ denotes the anion of a chiral acid. Wherein the compound according to formula 5 is preferably enriched in one of the possible enantiomers.

In the process according to Aspect 2 of the invention the chiral acid may be selected from among: (1S,4R)-camphor-10-sulphonic acid, (1R,4S)-camphor-10-sulphonic acid, (2R,3R)-di-p-toluoyltartaric acid, (2S,3S)-di-p-toluoyltartaric acid, (2R,3R)-tartaric acid, (2S,3S)-tartaric acid, (2R,3R)-dibenzoyltartaric acid and (2S,3S)-dibenzoyltartaric acid, more preferably (1S,4R)-camphor-10-sulphonic acid, (1R,4S)-camphor-10-sulphonic acid, (2R,3R)-di-p-toluoyltartaric acid, (2S,3S)-di-p-toluoyltartaric acid, (2R,3R)-tartaric acid, (2S,3S)-tartaric acid and (2R,3R)-dibenzoyltartaric acid (Variant 2.1).

In the process according to Aspect 2 of the invention and also Variant 2.1 the chiral acid may be placed in a solvent or mixture of solvents and to this is added a concentrated toluene solution of the compound of formula 5 in which the compound of formula 5 is preferably present in an amount of at least 60 wt. % (Variant 2.2).

In the process according to Aspect 2 of the invention and also in Variants 2.1 and 2.2 the solvent may be selected from among polar protic or aprotic solvents, preferably acetone, C₁- to C₅-alcohols, C₂- to C₃-nitriles, C₃- to C₆-ketones, with or without the addition of water (Variant 2.3).

In the process according to Aspect 2 of the invention and also in Variants 2.1, 2.2 and 2.3 the chiral acid may be dissolved in the solvent with heating to a temperature in the range from about 35° C. to approximately the reflux temperature of the solvent used (Variant 2.4).

In the process according to Aspect 2 of the invention and also in Variants 2.1, 2.2, 2.3 and 2.4 the concentrated toluene solution of the compound of formula 5 may be added to the solution of the chiral acid at or close to the dissolution temperature of the chiral acid (Variant 2.5).

In the process according to Aspect 2 of the invention and also in Variants 2.1 to 2.5 cooling may be carried out after the addition of the concentrated toluene solution and optional heating to the reflux temperature of the solvent (Variant 2.6).

In the process according to Aspect 2 of the invention and also in Variants 2.1 to 2.6 the compound of formula 5′ may be precipitated at a final temperature of between −15 and 35° C., preferably 5 to 35° C. (Variant 2.7).

In the process according to Aspect 2 of the invention and also in Variants 2.1 to 2.7 the precipitation may be assisted by inoculation with a small amount of seed crystals and/or trituration (Variant 2.8).

In the process according to Aspect 2 of the invention and also in Variants 2.1 to 2.8 the compound of formula 5′ may be precipitated in the form of an enantiomer in an enantiomeric purity of more than about 95%, preferably more than about 96%, particularly preferably more than about 98%, particularly more than about 99%, most particularly preferably more than about 99.9% (Variant 2.9).

A third aspect of the invention relates to an enantiomerically pure salt of the compound of formula 5 with a chiral acid.

A fourth aspect of the invention relates to a chiral, preferably enantiomerically pure salt of the compound of formula 5 with a chiral acid, which is crystalline.

The salt according to one of aspects 3 or 4 of the invention preferably excludes (1R,5S)-anhydroecgonin ethyl ester-(2′S,3′S)-dibenzoylhydrogen tartrate.

In the salt according to one of aspects 3 or 4 of the invention the chiral acid is preferably selected from: (1S,4R)-camphor-10-sulphonic acid, (1R,4S)-camphor-10-sulphonic acid, (2R,3R)-di-p-toluoyltartaric acid, (2S,3S)-di-p-toluoyltartaric acid, (2R,3R)-tartaric acid, (2S,3S)-tartaric acid, (2R,3R)-dibenzoyltartaric acid and (2S,3S)-dibenzoyltartaric acid.

In the salt according to one of aspects 3 or 4 of the invention the chiral acid is preferably selected from: (1S,4R)-camphor-10-sulphonic acid, (1R,4S)-camphor-10-sulphonic acid, (2R,3R)-di-p-toluoyltartaric acid, (2S,3S)-di-p-toluoyltartaric acid, (2R,3R)-tartaric acid, (2S,3S)-tartaric acid, (2R,3R)-dibenzoyltartaric acid.

In the salt according to one of aspects 3 or 4 of the invention, including all the above-mentioned variants regarding the chiral acid, the compound of formula 5 is preferably selected from: (1R,5S)-anhydroecgonin ethyl ester, (1R,5S)-anhydroecgonin methyl ester, (1R,5S)-anhydroecgonin propyl ester and (1R,5S)-anhydroecgonin butyl ester.

The salt according to one of aspects 3 or 4 of the invention is preferably selected among:

(1R,5S)-anhydroecgonin ethyl ester-(1′S,4′R)-camphor-10-sulphonate;

(1R,5S)-anhydroecgonin ethyl ester-(1′R,4′S)-camphor-10-sulphonate;

(1R,5S)-anhydroecgonin ethyl ester-(2′S,3′S)-di-p-toluoylhydrogen tartrate;

(1R,5S)-anhydroecgonin ethyl ester-(2′R,3′R)-di-p-toluoylhydrogen tartrate;

(1R,5S)-anhydroecgonin ethyl ester-(2′R,3′R)-hydrogen tartrate-monohydrate;

(1R,5S)-anhydroecgonin ethyl ester-(2′S,3′S)-hydrogen tartrate-monohydrate;

(1R,5S)-anhydroecgonin ethyl ester-(2′R,3′R)-hydrogen tartrate;

(1R,5S)-anhydroecgonin ethyl ester-(2′S,3′S)-hydrogen tartrate;

(1R,5S)-anhydroecgonin ethyl ester-(2′R,3′R)-dibenzoylhydrogen tartrate;

(1R,5S)-anhydroecgonin ethyl ester-(2′S,3′S)-dibenzoylhydrogen tartrate.

The salt according to one of aspects 3 or 4 of the invention is preferably selected from among:

(1R,5S)-anhydroecgonin ethyl ester-(1′S,4′R)-camphor-10-sulphonate;

(1R,5S)-anhydroecgonin ethyl ester-(1′R,4′S)-camphor-10-sulphonate;

(1R,5S)-anhydroecgonin ethyl ester-(2′S,3′S)-di-p-toluoylhydrogen tartrate;

(1R,5S)-anhydroecgonin ethyl ester-(2′R,3′R)-di-p-toluoylhydrogen tartrate;

(1R,5S)-anhydroecgonin ethyl ester-(2′R,3′R)-hydrogen tartrate-monohydrate;

(1R,5S)-anhydroecgonin ethyl ester-(2′S,3′S)-hydrogen tartrate-monohydrate;

(1R,5S)-anhydroecgonin ethyl ester-(2′R,3′R)-hydrogen tartrate;

(1R,5S)-anhydroecgonin ethyl ester-(2′S,3′S)-hydrogen tartrate;

(1R,5S)-anhydroecgonin ethyl ester-(2′R,3′R)-dibenzoylhydrogen tartrate.

The salt according to one of aspects 3 or 4 of the invention, including all the above mentioned variants and preferences, preferably has an enantiomeric purity of more than about 95%, preferably more than about 96%, particularly preferably more than about 98%, particularly more than about 99%, most particularly preferably more than about 99.9%.

The salt according to one of aspects 3 or 4 of the invention, including all the above mentioned variants and preferences, may be a solvate, preferably a hydrate, more preferably a monohydrate.

A fifth aspect of the invention relates to a solution of a (1R,5S)-anhydroecgonin ester of general chemical formula 5 as described under the first aspect of the invention, in toluene, xylene (all the isomers), halobenzenes, aliphatic hydrocarbons (C₅ to C₈), halogen-containing, aliphatic hydrocarbons (C₁ to C₆), aliphatic ethers (C₄ to C₈), esters of formic acid (C₂ to C₇), esters of acetic acid (C₃ to C₇) or nitriles (C₂ to C₅).

The preferred solvent is toluene.

The content of (1R,5S)-anhydroecgonin ester is at least 20 wt. %, preferably at least 40 wt. % and more preferably at least 60 wt. %.

A sixth aspect of the invention relates to a suspension consisting of a suspension agent selected from among toluene, xylene (all the isomers), halobenzenes, aliphatic hydrocarbons (C₅ to C₈), halogen-containing, aliphatic hydrocarbons (C₁ to C₆), aliphatic ethers (C₄ to C₈), C₂-C₇-alkyl-esters of formic acid, esters of acetic acid (C₃ to C₇) or C₂-C₅-alkyl-nitriles, preferably toluene and a salt according to one of aspects 3 or 4 of the invention, including all the variants and preferences thereof.

The following Examples serve to illustrate some methods of synthesis carried out by way of example. They are intended solely as possible procedures described by way of example without restricting the invention to their contents.

### Examples

### Example 1

### Preparation of anhydroecgonin ethyl ester

The anhydroecgonin ethyl ester is prepared for example by transesterification, according to the literature, of the anhydroecgonin methyl ester prepared for example by the methods described hereinbefore, in accordance with the following chemical equation:

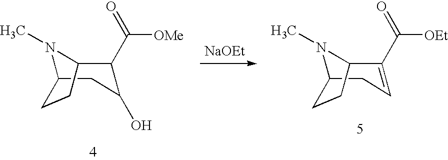

170 l of 2-carbomethoxytropinol concentrate (solvent: ethyl acetate; total alkaloid content calculated as 2-carbomethoxytropinol: 34.4 kg, determined by HClO₄-titration) are evaporated down at 52-58° C. in vacuo to a volume of 60 l and to this are added 350 l ethanol. The solution is cooled to 18° C. and at this temperature combined with 78.3 kg of 21% sodium ethoxide solution. The reaction mixture is heated to 52° C. with stirring for 1 h and then heated to 65° C. for 1 h. It is cooled to 18° C. and then combined with 34 l of glacial acetic acid. The reaction mixture is then evaporated down to 70 l at 51-58° C. in vacuo and 140 l of toluene are added. 410 l of condensate are added to the resulting solution and it is adjusted to a pH of 1.4 at 21° C. with 35 l of 50% sulphuric acid. The two phases are separated from each other and the toluene phase is discarded. The aqueous phase is combined with 340 l of toluene and adjusted to a pH of 8.5 with stirring at 24° C. using 54.5 l of 50% sodium hydroxide solution. The phases are separated and the aqueous phase is extracted again with 340 l of toluene. The combined toluene phases are combined with 5.1 kg of sodium sulphate and after 15 min 0.7 kg of activated charcoal and 0.34 kg of kieselguhr are added. After filtration has been carried out the solvent is distilled off in vacuo at 50-58° C. 30 l of toluene anhydroecgonin ethyl ester concentrate remain. Content of nitrogen bases (HClO₄ titration) 93.4%; enantiomeric purity (chiral HPLC) 4.5% area (1S,5R)-anhydroecgonin ethyl ester.

### Examples of the Preparation of Salts of (1R,5S)-anhydroecgonin ethyl ester

The reaction with chiral acids is illustrated by a number of Examples.

### Example 2

### Preparation of (1R,5S)-anhydroecgonin ethyl ester-(1′S,4′R)-camphor-10-sulphonate

The reaction is carried out according to the following chemical equation:

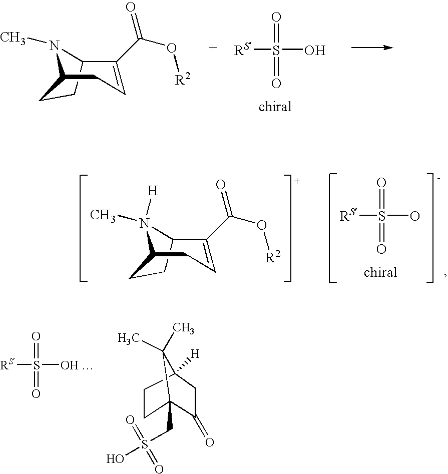

wherein R²=ethyl.

23.8 g (1S,4R)-camphor-10-sulphonic acid are dissolved in 120 ml acetone at 40° C. and 25.3 g toluene concentrate of anhydroecgonin ethyl ester [content of nitrogen bases (HClO₄ titration) 79.1%; chromatographic purity (GC, without toluene) 90.4% area anhydroecgonin ethyl ester; enantiomeric purity (chiral HPLC) 4.4% area (1S,5R)-anhydroecgonin ethyl ester] are added thereto with stirring. The mixture is evaporated down in vacuo at 40° C. and the oily residue remaining is taken up in 30 ml acetone at ambient temperature. Then a small amount of seed crystals are added (approx. 0.1 g), the mixture is cooled and the temperature is maintained at 5° C. for 15 h. The final temperature during the precipitation is preferably adjusted to between −15 and 10° C.

The resulting suspension is suction filtered through a Büchner funnel, the precipitate is washed with 20 ml cold acetone and then dried for 15 h at 50° C. in vacuo (p=approx. 30 mbar). The salt is precipitated from acetone in dilutions (Σ m_{educts}: Σ Vₛₒₗᵥₑₙₜ) of from 2:1 to 1:1. As washing liquid it is possible to use, apart from acetone, more lipophilic solvents or mixtures of solvents.

16.2 g of the camphor-10-sulphonate are obtained in the form of white crystals.

M.p. 136° C.; water content (Karl-Fischer titration) 0.1%; content of nitrogen bases (HClO₄ titration, based on anhydrous substance) 98.8%; chromatographic purity (GC, after liberation of bases) 98.8% Fl. Anhydroecgonin ethyl ester; enantiomeric purity (chiral HPLC) 99.9% area (1R,5S)-anhydroecgonin ethyl ester/0.1% Fl. (1S,5R)-anhydroecgonin ethyl ester.

### Example 3

### Preparation of (1R,5S)-anhydroecgonin ethyl ester-(2′S,3′S)-di-p-toluoylhydrogen tartrate

The reaction is carried out according to the following chemical equation:

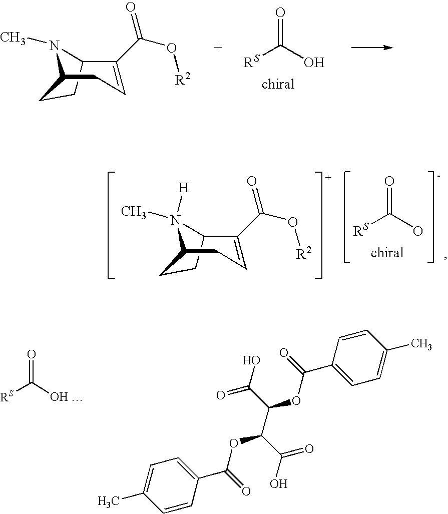

wherein R²=ethyl.

36.1 g (2S,3S)-di-p-toluoyltartaric acid are dissolved in 120 ml acetonitrile at 50° C., and 22.8 g toluene concentrate of anhydroecgonin ethyl ester [content of nitrogen bases (HClO₄ titration) 79.1%; chromatographic purity (GC, without toluene) 90.4% area anhydroecgonin ethyl ester; enantiomeric purity (chiral HPLC) 4.4% area (1S,5R)-anhydroecgonin ethyl ester] are rapidly added thereto with stirring. The solution is cooled to ambient temperature within 2 h with stirring. At the start of the cooling phase a small amount of seed crystals (approx. 0.1 g) are added. The final temperature during the precipitation is preferably between 5 and 35° C. The salt is particularly preferably precipitated from acetonitrile in dilutions (Σ m_{educts}: Σ ₛₒₗᵥₑₙₜ) from 1:1 to 1:3. Stirring is continued for 2 h and the precipitate formed is separated off by vacuum filtration. The precipitate is washed with 20 ml acetonitrile at ambient temperature and then dried for 15 h at 50° C. in vacuo (p=approx. 30 mbar). As washing liquid it is possible to use, instead of acetonitrile, more lipophilic solvents or mixtures of solvents. Conventional devices may be used for separating the precipitated solid and mother liquor, for example a suction filter, centrifuge, decanter, pressure filter etc. 41.3 g of the di-p-toluoylhydrogen tartrate are obtained in the form of a white solid. M.p. 142° C.; water content (Karl Fischer titration) 1.0%; content of nitrogen bases (HClO₄ titration, based on anhydrous substance) 100.4%; chromatographic purity (GC, after liberation of bases) 98.1% area anhydroecgonin ethyl ester; enantiomeric purity (chiral HPLC) 99.4% area (1R,5S)-anhydroecgonin ethyl ester/0.6% area (1S,5R)-anhydroecgonin ethyl ester.

### Example 4

### Preparation of (1R,5S)-anhydroecgonin ethyl ester-(2′R,3′R)-hydrogen tartrate-monohydrate

The reaction is carried out according to the following chemical equation:

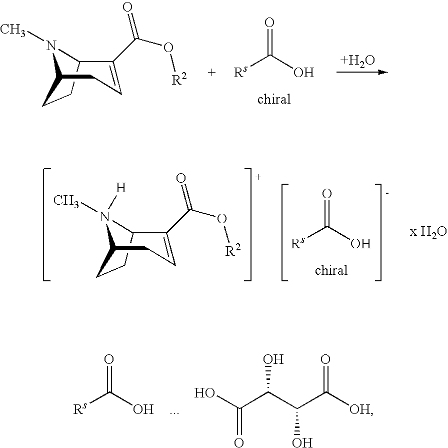

wherein R²=ethyl.

Batch A: 43.9 g (2R,3R)-tartaric acid are taken up in a mixture of 15 ml H₂O and 300 ml acetone, refluxed for 5 min and the resulting solution is filtered hot. 63.2 g of toluene concentrate of anhydroecgonin ethyl ester [content of nitrogen bases (HClO₄ titration) 79.1%; chromatographic purity (GC, without toluene) 90.4% area anhydroecgonin ethyl ester; enantiomeric purity (chiral HPLC) 4.4% area (1S,5R)-anhydroecgonin ethyl ester] are taken up in 200 ml acetone at ambient temperature, filtered and then added to the tartaric acid solution. The combined filtrates are refluxed for 10 min and cooled to 20° C. with stirring within 3 h. At the start of the cooling phase a small amount of seed crystals (approx. 0.1 g) is added. The mixture is stirred for another 2 h at 20° C. and the crystals precipitated are separated off by vacuum filtration. The final temperature during the precipitation is preferably between 5 and 35° C. The salt is preferably precipitated from acetone/H₂O in dilutions (Σ m_{educts}: Σ Vₛₒₗᵥₑₙₜ) of 1:2 to 1:8, solvent ratio of acetone/H₂O 10:0.1 to 10:1. The precipitated solid and mother liquor are separated using conventional apparatus, such as a suction filter, centrifuge, decanter, pressure filter etc. If necessary, filtering and suction compounds may be used for filtration of the educts. The crystals are washed twice with 100 ml acetone at ambient temperature and then dried for 15 h at 50° C. in vacuo (p=approx. 30 mbar). 73.9 g of the hydrogen tartrate salt are obtained in the form of a monohydrate. M.p. 86° C.; water content (Karl Fischer titration) 5.1%; content of nitrogen bases (HClO₄ titration, based on anhydrous substance) 99.9%; chromatographic purity (GC, after liberation of bases) 99.0% area anhydroecgonin ethyl ester; enantiomeric purity (chiral HPLC) 99.8% area (1R,5S)-anhydroecgonin ethyl ester/0.2% area (1S,5R)-anhydroecgonin ethyl ester.

Batch B: 80.0 g (2R,3R)-tartaric acid are taken up in a mixture of 600 ml acetone and 40 ml H₂O at ambient temperature and the resulting solution is filtered. The filter is washed with 10 ml acetone and the filtrate is heated to 55° C. 126.4 g of toluene concentrate of anhydroecgonin ethyl ester [content of nitrogen bases (HClO₄ titration) 79.1%; chromatographic purity (GC, without toluene) 90.4% area anhydroecgonin ethyl ester; enantiomeric purity (chiral HPLC) 4.4% area (1 S,5R)-anhydroecgonin ethyl ester] are taken up in 400 ml acetone at ambient temperature, filtered and then added to the heated tartaric acid solution. The combined filtrates are refluxed for 10 min and then cooled to 20° C. with stirring within 3 h. At the start of the cooling phase a small amount of seed crystals (approx. 0.1 g) is added. The mixture is stirred for a further 72 h at 20° C. and the precipitated crystals are separated off by vacuum filtration. The final temperature during the precipitation is preferably between 5 and 35° C. The salt is preferably precipitated from acetone/H₂O in dilutions (Σ m_{educts}: Σ Vₛₒₗᵥₑₙₜ) of 1:2 to 1:8, solvent ratio of acetone/H₂O 10:0.1 to 10:1. The separation of the precipitated solid and mother liquor is carried out using conventional apparatus, such as a suction filter, centrifuge, decanter, pressure filter etc. If necessary, filtering and suction compounds may be used for filtration of the educts. The crystals are washed twice with 200 ml acetone at ambient temperature and then dried for 15 h at 50° C. in vacuo (p=30 mbar). 150.7 g of the hydrogen tartrate monohydrate are obtained in the form of white crystals. M.p. 85° C.; water content (Karl Fischer titration) 5.3%; content of nitrogen bases (HClO₄ titration, based on anhydrous substance) 100.3%; chromatographic purity (GC, after liberation of bases) 99.0% area anhydroecgonin ethyl ester; enantiomeric purity (chiral HPLC) 99.8% area (1R,5S)-anhydroecgonin ethyl ester/0.2% area (1 S,5R)-anhydroecgonin ethyl ester.

### Example 5

### Preparation of (1R,5S)-anhydroecgonin ethyl ester-(2′R,3′R)-dibenzoylhydrogen tartrate

The reaction is carried out according to the following chemical equation:

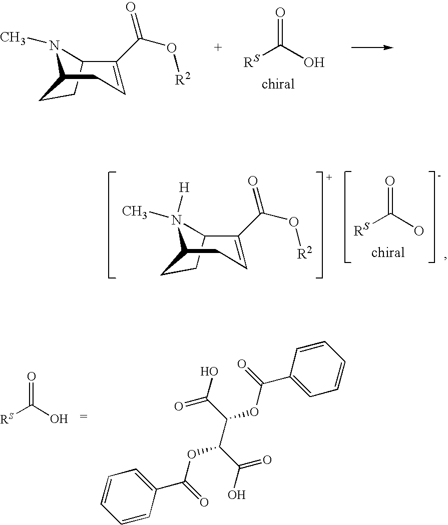

wherein R²=ethyl.

83.5 g (2R,3R)-dibenzoyltartaric acid are refluxed in 300 ml of ethanol for 30 min. 48.9 g toluene concentrate of anhydroecgonin ethyl ester [content of nitrogen bases (HClO₄ titration) 93.1%; chromatographic purity (GC, without toluene) 94.7% area anhydroecgonin ethyl ester; enantiomeric purity (chiral HPLC) 4.5% area (1S,5R)-anhydroecgonin ethyl ester] are added to the resulting clear solution with stirring and the solution is refluxed for 5 min. Then it is left to cool to 20° C. within 2 h with stirring, during which time a precipitate settles out. The suspension is stirred for a further 2 h at 20° C. and then suction filtered through a Büchner funnel. The final temperature during the precipitation is preferably between 5 and 35° C. The precipitation of the salt from ethanol is preferably carried out in dilutions (Σ m_{educts}: Σ Vₛₒₗᵥₑₙₜ) of 1:1 to 1:6. The separation of the precipitated solid and mother liquor is carried out using conventional apparatus, such as, for example, a suction filter, centrifuge, decanter, pressure filter etc. The precipitate is washed twice with 50 ml ethanol at ambient temperature and then dried for 15 h at 50° C. in vacuo (p=approx. 30 mbar). 120.1 g of the dibenzoylhydrogen tartrate are obtained in the form of a white solid. M.p. 149° C., water content (Karl Fischer titration) 0.2%; content of nitrogen bases (HClO₄ titration, based on anhydrous substance) 99.8%; chromatographic purity (GC, after liberation of bases) 98.2% area anhydroecgonin ethyl ester; enantiomeric purity (chiral HPLC) 97.3% area (1R,5S)-anhydroecgonin ethyl ester/2.7% area (1S,5R)-anhydroecgonin ethyl ester.

1. A process for preparing a chiral salt of a (1R,5S)-anhydroecgonin ester of the formula 5: 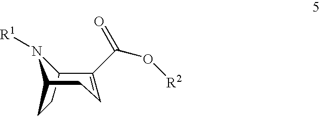
wherein
R¹ denotes hydrogen, an alkyl group which is methyl, ethyl, propyl or butyl, or a protective group which is allyl, benzyl, methoxybenzyl, allyloxycarbonyl, benzyloxycarbonyl, tert.-butyloxycarbonyl, 9-fluorenylmethyloxycarbonyl, acetyl, benzoyl or formyl;
R² denotes alkyl, aryl which is phenyl or naphthyl, optionally substituted by one or more substituents which are each halogen, hydroxy, amino, cyano, nitro, trifluoromethyl, trifluoromethoxy, alkoxy, cycloalkoxy, alkyl, cycloalkyl, cycloalkylalkyl, alkenyl and alkynyl; preferably R² is methyl, ethyl, propyl or butyl:
comprising the steps of
(1a) reacting a compound of formula 1 with methanol and optionally in methanol as solvent to form a compound of formula 1′: 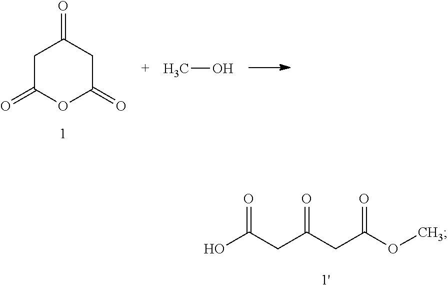
(1b) reacting a compound of formula 2 with water and catalyst to form a compound of formula 2′: 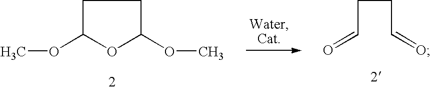
(2) reacting a compound of formula 1′ and a compound of formula 2′ with an R¹-amine solution to form a compound of formula 3: 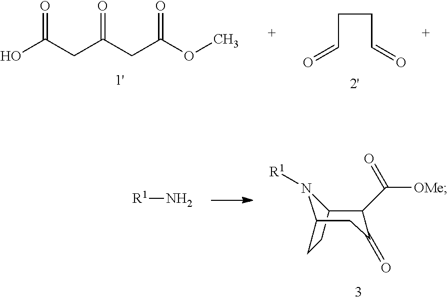
wherein R¹ denotes hydrogen, an alkyl group which is methyl, ethyl, propyl or butyl, or a protective group which is allyl, benzyl, methoxybenzyl, allyloxycarbonyl, benzyloxycarbonyl, tert.-butyloxycarbonyl, 9-fluorenylmethyloxycarbonyl, acetyl, benzoyl or formyl;
(3) optionally reacting the compound of formula 3 with (2R,3R)-tartaric acid [L(+)-tartaric acid] with enrichment of the (1R,5S)-enantiomer-hydrogen tartrate salt of the compound of formula 3′: 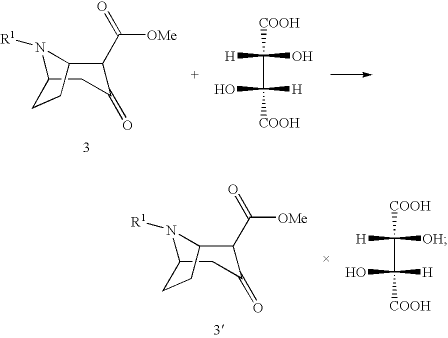
(4) optionally releasing compound 4 in the basic 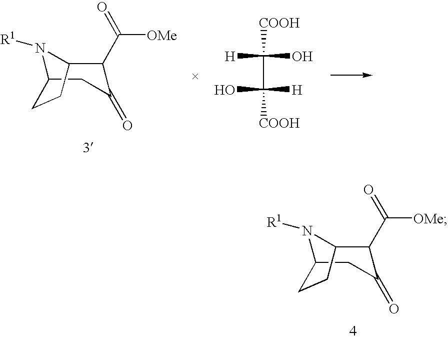
(5) reducing the product obtained and optionally carrying out further transesterification with an alkoxide of formula MOR² to obtain a compound of formula 5: 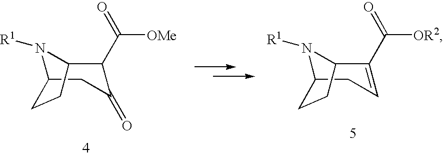
wherein R² is as hereinbefore defined and M denotes an alkali or alkaline earth metal, preferably potassium or sodium; and
(6) reacting the compound of formula 5 with a chiral acid which is (1S,4R)-camphor-10-sulphonic acid, (1R,4S)-camphor-10-sulphonic acid, (2R,3R)-di-p-toluoyltartaric acid, (2S,3S)-di-p-toluoyltartaric acid, (2R,3R)-tartaric acid, (2S,3S)-tartaric acid, (2R,3R)-dibenzoyltartaric acid or (2S,3S)-dibenzoyltartaric acid, to form a salt of the compound of formula 5′; 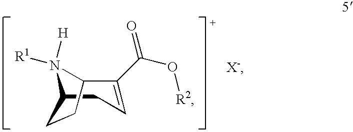
wherein X⁻ denotes the anion of chiral acid;
(7) optionally purifying the chiral compound of formula 5′ and
(8) optionally carrying out crystallisation.

2. The process according to claim 1, characterised in that at the end of the working up in step (5) a concentrated toluene solution of the compound of formula 5 is prepared by the addition of toluene.

3. The process according to claim 1, characterised in that in step (6) the compound of formula 5 is present as a concentrate dissolved in toluene in an amount of at least 20 wt.

4. The process according to claim 1, characterised in that in step (6) the chiral acid is placed in a solvent and to this is added the concentrated toluene solution of the compound of formula 5.

5. The process according to claim 1, characterised in that the solvent in step (6) is acetone, a C₁- to C₅-alcohol, a C₂- to C₃-nitrile, or a C₃- to C₆-ketone, with or without the addition of water.

6. The process according to claim 1, characterised in that the chiral acid is dissolved in the solvent in step (6) with heating to a temperature in the range from about 35° C. to approximately the reflux temperature of the solvent used, the compound of formula 5 is added in the form of a concentrated toluene solution or suspension to the solution of the chiral acid at or around the temperature of dissolution of the chiral acid, after the addition of the concentrated toluene solution or suspension and optionally heating to the reflux temperature of the solvent, in order to precipitate the compound of formula 5′ the mixture is cooled to a final temperature of between −15 and 35° C.

7. A process for preparing a salt of a compound of the formula 5: 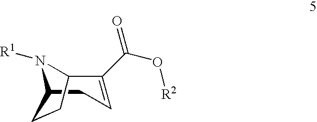
wherein the compound of formula 5 is reacted with a chiral acid to form a salt of the compound of formula 5′: 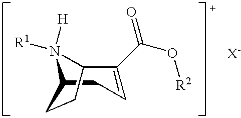
wherein
R¹ denotes hydrogen, an alkyl group which is methyl, ethyl, propyl or butyl, or a protective group which is allyl, benzyl, methoxybenzyl, allyloxycarbonyl, benzyloxycarbonyl, tert.-butyloxycarbonyl, 9-fluorenylmethyloxycarbonyl, acetyl, benzoyl or formyl;
R² denotes alkyl, aryl which is phenyl or naphthyl, optionally substituted by one or more substituents which are each halogen, hydroxy, amino, cyano, nitro, trifluoromethyl, trifluoromethoxy, alkoxy, cycloalkoxy, alkyl, cycloalkyl, cycloalkylalkyl, alkenyl and alkynyl; preferably R² is methyl, ethyl, propyl or butyl; and
X⁻ denotes the anion of the chiral acid which is (1S,4R)-camphor-10-sulphonic acid, (1R,4S)-camphor-10-sulphonic acid, (2R,3R)-di-p-toluoyltartaric acid, (2S,3S)-di-p-toluoyltartaric acid, (2R,3R)-tartaric acid, (2S,3S)-tartaric acid, (2R,3R)-dibenzoyltartaric acid or (2S,3S)-dibenzoyltartaric acid.

8. (canceled)

9. The process according to claim 7, characterised in that the compound of formula 5′ is precipitated in the form of an enantiomer in an enantiomeric purity of more than about 95%.

10. An enantiomerically pure salt of a compound of the formula 5 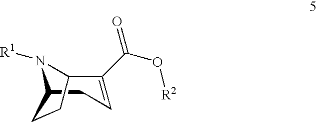
wherein
R¹ denotes hydrogen, an alkyl group which is methyl, ethyl, propyl or butyl, or a protective group which is allyl, benzyl, methoxybenzyl, allyloxycarbonyl, benzyloxycarbonyl, tert.-butyloxycarbonyl, 9-fluorenylmethyloxycarbonyl, acetyl, benzoyl or formyl;
R² denotes alkyl, aryl which is phenyl or naphthyl, optionally substituted by one or more substituents which are each halogen, hydroxy, amino, cyano, nitro, trifluoromethyl, trifluoromethoxy, alkoxy, cycloalkoxy, alkyl, cycloalkyl, cycloalkylalkyl, alkenyl and alkynyl; preferably R² is methyl, ethyl, propyl or butyl;
with a chiral acid which is (1S,4R)-camphor-10-sulphonic acid, (1R,4S)-camphor-10-sulphonic acid, (2R,3R)-di-p-toluoyltartaric acid, (2S,3S)-di-p-toluoyltartaric acid, (2R,3R)-tartaric acid, (2S,3S)-tartaric acid, (2R,3R)-dibenzoyltartaric acid or (2S,3S)-dibenzoyltartaric acid,
which enantiomerically pure salt is not (1R,5S)-anhydroecgonin ethyl ester-(2′S,3′S)-dibenzoylhydrogen tartrate.

11. (canceled)

12. A salt according to claim 10 which is (1R,5S)-anhydroecgonin ethyl ester, (1R,5S)-anhydroecgonin methyl ester, (1R,5S)-anhydroecgonin propyl ester or (1R,5S)-anhydroecgonin butyl ester.

13. A salt according to claim 10 which is selected from the group consisting of
(1R,5S)-anhydroecgonin ethyl ester-(1′S,4′R)-camphor-10-sulphonate;
(1R,5S)-anhydroecgonin ethyl ester-(1′R,4′S)-camphor-10-sulphonate;
(1R,5S)-anhydroecgonin ethyl ester-(2′S,3′S)-di-p-toluoylhydrogen tartrate;
(1R,5S)-anhydroecgonin ethyl ester-(2′R,3′R)-di-p-toluoylhydrogen tartrate;
(1R,5S)-anhydroecgonin ethyl ester-(2′R,3′R)-hydrogen tartrate-monohydrate;
(1R,5S)-anhydroecgonin ethyl ester-(2′S,3′S)-hydrogen tartrate-monohydrate;
(1R,5S)-anhydroecgonin ethyl ester-(2′R,3′R)-hydrogen tartrate;
(1R,5S)-anhydroecgonin ethyl ester-(2′S,3′S)-hydrogen tartrate;
(1R,5S)-anhydroecgonin ethyl ester-(2′R,3′R)-dibenzoylhydrogen tartrate,
(1R,5S)-anhydroecgonin ethyl ester-(2′S,3′S)-dibenzoylhydrogen tartrate, preferably
(1R,5S)-anhydroecgonin ethyl ester-(1′S,4′R)-camphor-10-sulphonate;
(1R,5S)-anhydroecgonin ethyl ester-(1′R,4′S)-camphor-10-sulphonate;
(1R,5S)-anhydroecgonin ethyl ester-(2′S,3′S)-di-p-toluoylhydrogen tartrate;
(1R,5S)-anhydroecgonin ethyl ester-(2′R,3′R)-di-p-toluoylhydrogen tartrate;
(1R,5S)-anhydroecgonin ethyl ester-(2′R,3′R)-hydrogen tartrate-monohydrate;
(1R,5S)-anhydroecgonin ethyl ester-(2′S,3′S)-hydrogen tartrate-monohydrate;
(1R,5S)-anhydroecgonin ethyl ester-(2′R,3′R)-hydrogen tartrate;
(1R,5S)-anhydroecgonin ethyl ester-(2′S,3′S)-hydrogen tartrate;
(1R,5S)-anhydroecgonin ethyl ester-(2′R,3′R)-dibenzoylhydrogen tartrate.
(1R,5S)-anhydroecgonin ethyl ester-(2′S,3′S)-hydrogen tartrate; and
(1R,5S)-anhydroecgonin ethyl ester-(2′R,3′R)-dibenzoylhydrogen tartrate.

14-15. (canceled)
